(19) United States
(12) Patent Application Publication
(10) Pub. No.: US 2012/0225069 A1
(43) Pub. Date: Sep. 6, 2012

(54) USE OF A COMPOUND FOR REDUCING THE BIOLOGICAL EFFECTIVENESS OF IL-6

(76) Inventors: AHMED SHERIFF, Berlin (DE); Birgit Vogt, Berlin (DE)

(21) Appl. No.: 13/412,716

(22) Filed: Mar. 6, 2012

Related U.S. Application Data
(63) Continuation of application No. 12/585,545, filed on Sep. 17, 2009, now abandoned.
Application No. 12/585,545 is a division of application No. 10/573,049, filed on Jun. 21, 2006, now abandoned.

(30) Foreign Application Priority Data
Sep. 22, 2003 (DE) 103 44 206.5

(51) Int. Cl. A61K 39/395; A61P 9/00; A61P 9/10; A61P 1/16; A61P 1/04; A61P 37/00; A61P 35/02; A61P 37/06; A61P 19/02; A61P 29/00; A61P 5/00

(52) U.S. Cl. 424/135.1; 424/158.1; 424/173.1; 424/144.1; 424/145.1; 424/136.1

(57) ABSTRACT

Use of a compound comprising at least a structural entity which binds or is an antagonist for interleukin-6 (IL-6) and/or the IL-6 receptor or parts of it, preferably human IL-6 which compound depletes IL-6 from a solution or blocks at least one or more IL-6 functions on cell surfaces or in a solution for manufacturing of a medicament for the treatment or prevention of diseases selected from the group consisting of endothelial injury, destruction, increased risk for endothelial injury or destruction or immune disorders other than rheumatoid arthritis and combinations thereof.

The current invention relates to the use of a compound for decreasing levels of interleukin 6 (IL-6) and/or the unoccupied IL-6 receptor concentration in humans comprising administering to a mammal in need thereof an effective amount of a compound containing a molecule that binds IL-6 and/or the IL-6 receptor or a pharmaceutical salt or solvate thereof.

The present invention deals with the disciplines of therapeutic proteins, cardiovascular physiology, and pharmacology. Specifically, the present invention is related to decreasing known risk factors of e.g. cardiovascular disease and other related diseases with endothelial participation associated with increased levels of interleukin 6 (IL-6) by administering molecules that bind IL-6 and/or the IL-6 receptor.

Cardiovascular disease is a major cause of death in the United States and a major source of morbidity, medical cost, and economic loss to millions of people. Two of the most common and destructive aspects of cardiovascular disease are the appearance of arteriosclerosis and thrombolitic events.

In recent years, a great deal of progress has been achieved in the treatment of cardiovascular disease. This progress has been possible not only because of the advancement of therapeutic intervention in the disease mechanisms, but also through the early identification of patients at risk of developing the disease. Indeed, patient risk identification and early treatment are important features of modern medical practice. Over the last twenty years, a variety of factors and clinical parameters have been identified which correlate with either the current state or the future probability of developing cardiovascular disease. Such risk factors may include measurable biochemical or physiological parameters, e.g., serum cholesterol, HDL, LDL, fibrinogen levels, etc., or behavioural of life-style patterns, such as obesity, smoking, etc. The risk factor most germane to the present invention is the level of C-reactive protein. CRP is induced by IL-6.

The intrinsic relationship between a measurable parameter or risk factor and the disease state is not always clear. In other words, it is not always clear whether the risk factor itself is causative or contributory to the disease or is instead an ancillary reflection that is indicative of the disease. Thus, a therapeutic modality, which effects a risk factor, may be directly modifying a pathological mechanism of the disease and its future course, or may be indirectly benefiting some contributory process related to the disease.

Additionally, many risk factors associated with cardiovascular disease are involved in other pathological states in either a causative or indicative role. Therefore, reduction or blockade of a particular risk factor in cardiovascular disease may have other beneficial effects in other diseases related to that risk factor.

Of particular interest to the methods of the present invention is the reduction of cardiovascular risk factors associated with abnormally high levels of C-reactive protein.

C-reactive protein is produced by the liver in response to IL-6 production. IL-6 is produced as part of an inflammatory response in the body. Thus, C-reactive protein as well as IL-6 levels are markers of systemic inflammatory activity. Chronic inflammation is thought to be one of the underlying and sustaining pathologies in cardiovascular disease.

At menopause, with the loss of estrogen, women's prevalence of cardiovascular disease increases. Also, the risk factors of cardiovascular disease increase, especially lipid (cholesterol and triglyceride), homocysteine, and C-reactive protein levels. Today, the most common method of preventing cardiovascular disease in post-menopausal women is Hormone Replacement Therapy (HRT). However, many women do not comply with this therapy because of the unpleasant side-effects, such as bloating, resumption of mensus, breast tenderness, fear of uterine and breast cancer, etc. Additionally, while HRT does lower cholesterol and homocysteine levels, HRT raises C-reactive protein and IL-6 levels. An object of the invention is to provide a therapeutic agent which lowers these risk factors.

Another object of the present invention is to provide tools, molecules and methods for decreasing levels of IL-6 in humans. This object is solved by the use of a compound comprising at least a structural entity which binds or is an antagonist for interleukin-6 (IL-6) and/or the IL-6 receptor or parts of it, preferably human IL-6 and/or the human IL-6 receptor which compound depletes IL-6 from a solution or blocks at least one or more IL-6 functions on cell surfaces or in a solution for manufacturing of a medicament for the treatment or prevention of diseases selected from the group consisting of endothelial injury, destruction, increased risk for endothelial injury or destruction or immune disorders other than rheumatoid arthritis and combinations thereof.

Further, the present invention relates to a method for inhibiting conditions or detrimental effects caused by an excess of IL-6, respectively comprising administering to a human in need thereof, an effective amount of a compound containing at least a molecule which binds interleukin-6 (IL-6) and/or the IL-6 receptor or a pharmaceutical salt or solvate thereof.

The present invention is based to the finding that molecules that bind interleukin-6 (IL-6) and/or the IL-6 receptor, i.e., antibodies, a recombinant antibody (as e.g. single chain antibody—scAb or scFv; bispecific antibody, diabody), monoclonal antibodies, are useful for lowering the levels of IL-6 or blocking IL-6 and/or the IL-6 receptor.

As used herein, the term “effective amount” means an amount of a compound of molecules which bind IL-6 and/or the IL-6 receptor which is capable of decreasing levels of IL-6 or blocking IL-6 and/or the IL-6 receptor and/or inhibiting conditions or detrimental effects caused by an excess of IL-6, respectively.

The term “estrogen deficient” refers to a condition, either naturally occurring or clinically induced, where a woman can not produce sufficient estrogenic hormones to maintain estrogen dependent functions, e.g., menses, homeostasis of bone mass, neuronal function, cardiovascular condition, etc. Such estrogen deficient situations arise from, but are not limited to, menopause and surgical or chemical ovarectomy, including its functional equivalent, e.g., medication with GnRH agonists or antagonists, ICI 182780, and the like.

The term “inhibiting” in the context of inhibiting conditions or detrimental effects caused by an excess of IL-6 includes its generally accepted meaning, i.e., blocking, prohibiting, restraining, alleviating, ameliorating, slowing, stopping, or reversing the progression or severity of an increase of IL-6 and the pathological sequelae, i.e., symptoms, resulting from that event.

The term “pharmaceutical” when used herein as an adjective, means substantially non-toxic and substantially non-deleterious to the recipient.

By “pharmaceutical formulation” or “medicament” or “pharmaceutical composition” it is further meant that the carrier, solvent, excipients and salt must be compatible with the active ingredient of the formulation (a compound of at least a molecule, which binds IL-6 and/or the IL-6 receptor).

The term “solvate” represents an aggregate that comprises one or more molecules of the solute, with one or more molecules of a pharmaceutical solvent, such as water, buffer, physiological salt solution, and the like.

The objects underlying the present invention are in particular accomplished by the use of a compound comprising at least a structural entity which binds or is an antagonist for IL-6 and/or the IL-6 receptor or parts of it, preferably human IL-6 and which compound
a.) blocks at least one or more IL-6 functions on cell surfaces or in a solution, preferably blood or other body fluids or from tissues, most preferably in vivo for use in patients with acute endothelial injury and/or destruction, preferably for stroke, cardiac infarction, avoidance of sudden cardiac death, for burnt offering, for severe surgery or other injuries with severe wound areas, for diabetic shock, for acute liver failure, neurodegenerative diseases, for leukemic persons after irradiation and for long term endothelial injury and/or destruction, and for patients with atherosclerosis, with unstable angina, with diabetes type I or type II, with excessive body weight and/or obesity, for alcoholics, under Hormone Replacement Therapy (HRT), for old persons, for smokers and for preventing allograft transplant rejection or xeno-transplant rejection and for the induction of allo-transplant or xeno-transplant tolerance or inhibition of T cell activation and for preventing or treatment of autoimmune diseases other than rheumatoid arthritis, autoimmune liver disease and pancreatitis, and/or
b.) depletes IL-6 from a solution, preferably blood or other body fluids or from tissues, most preferably in vivo for use in patients with acute endothelial injury and/or destruction, preferably for stroke, cardiac infarction, avoidance of sudden cardiac death, for burnt offering, for severe surgery or other injuries with severe wound areas, for diabetic shock, for acute liver failure, neurodegenerative diseases, for leukemic persons after irradiation and for long term endothelial injury and/or destruction, preferably for patients with atherosclerosis, with unstable angina, with diabetes type I or type II, with overweight and/or obesity, for alcoholics, under Hormone Replacement Therapy (HRT), for old persons, for smokers and for preventing allograft transplant rejection or xeno-transplant rejection and for the induction of allo-transplant or xeno-transplant tolerance or inhibition of T cell activation and for preventing or treatment of autoimmune diseases other than rheumatoid arthritis, autoimmune liver disease and pancreatitis.

In one embodiment the compound of the invention is a polypeptide comprising a binding site to IL-6 and/or the IL-6 receptor, preferably an antibody containing an antigen-binding site to IL-6 and/or the IL-6 receptor. The compound of the invention is in particular a poly- or monoclonal antibody comprising an antigen-binding site to IL-6 and/or the IL-6 receptor.

The monoclonal antibody comprises particularly an antigen-binding site to IL-6 and/or the IL-6 receptor and is obtainable after immunizing vertebrates, preferably mammals such as mice, rats, guinea pigs, hamsters, monkeys, pigs, goats, chicken, cows, horses and rabbits. The poly- or monoclonal antibody comprising an antigen-binding site to IL-6 and/or the IL-6 receptor is preferably humanized according to technologies well-known to the skilled person. The compound of the invention can also be prepared by immunizing humanized mice and/or immune defective mice (as e.g. SCID or nude mice) repopulated with vital immune cells (e.g. of human origin; as e.g. SCID-hu mice).

In a further embodiment the antibody of the invention is a recombinant antibody (as e.g. single chain antibody—scAb or scFv; bispecific antibody, diabody etc.) capable of binding to IL-6 and/or the IL-6 receptor, in particular by containing the antigen-binding site of an antibody which is cross-reactive with IL-6 and/or the IL-6 receptor. The antibody molecule of the invention is a humanized or human antibody. Subject matter of the invention is also a host cell, preferably a stable host cell, producing the compound of the invention.

Furthermore, subject matter of the invention is at least one recombinant vector comprising the nucleotide sequences encoding the binding molecule fragments according to the invention, operably linked to regulating sequences capable of expressing the antibody molecule in a host cell, preferably as a secretory protein.

Subject matter of the present invention is also a host comprising, preferably stably transgenic, the vector according to the invention, a prokaryotic or eukaryotic cell line producing a recombinant antibody of the invention as well as a eukaryotic organism, most preferably an animal, a plant or a fungus, producing a recombinant antibody according to the invention.

Subject matter of the invention is also a method of producing a recombinant molecule of the invention capable of binding to the IL-6 and/or the IL-6 receptor antigen, comprising culturing a host cell and isolating the binding molecule from the culture medium and/or the producing cell.

In another embodiment, the present invention is related with a method for inhibiting immunologic, inflammatory and/or pathophysiological responses by treating patients with increased IL-6 levels with the IL-6 and/or the IL-6 receptor-binding molecules according to the invention.

Another subject of the present invention is a pharmaceutical composition for reducing the IL-6 concentration and/or the unoccupied IL-6 receptor concentration, containing a therapeutically effective amount of the binding molecule according to the invention and a pharmaceutically acceptable carrier. In addition to these compounds the medicament may comprise anti-inflammatory substances which are selected from the group consisting of C-reactive Protein (CRP) antagonists, CRP binding molecules, anti-IL-1β-molecules, PLA2 antagonists, PLA2 binding molecules, complement blockers or combinations thereof.

Still another embodiment of the invention is a method for reducing inflammatory immune and/or pathophysiological responses by reducing the IL-6 concentration and/or the unoccupied IL-6 receptor concentration, a method for reducing endothel injury and/or destruction by reducing the IL-6 concentration and/or the unoccupied IL-6 receptor concentration, a method for acute treatments in case of acute endothelial injury and/or destruction, preferably for stroke, cardiac infarction, avoidance of sudden cardiac death, for burnt offering, for severe surgery or other injuries with severe wound areas, for diabetic shock, for acute liver failure, for pancreatitis, neurodegenerative diseases, for leukemic persons after irradiation, a method for continuous treatments in case of long term endothelial injury and/or destruction, with atherosclerosis, with unstable angina, with diabetes type I or type II, with excessive body weight and/or obesity, for alcoholics, for persons under Hormone Replacement Therapy (HRT), for old persons, for smokers, a method for preventing allograft transplant rejection or xeno-transplant rejection, a method for the induction of allo-transplant or xeno-transplant tolerance or inhibition of T cell activation, and a method for preventing or treatment of autoimmune diseases other than rheumatoid arthritis, the methods comprising administering to a patient in need of such treatment a therapeutically effective amount of a pharmaceutical composition of the invention.

The compound of the invention can be combined with other molecules, preferably therapeutics for the respective disease or other anti-inflammatory molecules like e.g. C-reactive Protein (CRP) antagonists, CRP binding molecules, anti-IL-1β-molecules, anti-IL-1β receptor molecules, PLA2 antagonists, PLA2 binding molecules, and/or complement blockers.

The methods provided by the current invention are useful in both the treatment and prevention of harmful sequelae associated with elevated levels of IL-6. Since IL-6 serum concentration is related to levels and production of cytokines, which are especially produced in inflammatory processes, the methods of the current invention are useful in treating or preventing inflammatory events and sequelae, thereof. Such inflammatory events include, but are not limited to: arthritis (osteo), arterial and venous chronic inflammation, autoimmune diseases, e.g., SLE, multiple sclerosis, myasthenia gravis, Graves' disease, psoriasis vulgaris, dilated cardiomyopathy, diabetes mellitus, Bechterew, inflammatory bile disease, ulcerative colitis, Crohn's disease, idiopathic thrombocytopenia purpura (ITP), aplastic anemia, idiopathic dilated cardiomyopathy (IDM), autoimmune thyroiditis, Goodpastures disease and the like.

Methods of the current invention are useful for treating or preventing pathologic sequelae of atherosclerotic or thrombotic disease. Such pathologies include, but are not limited to stroke, circulatory insufficiency, ischemic events, myocardial infarction, pulmonary thromboembolism, stable and unstable angina, coronary artery disease, sudden death syndrome, and the like.

The present invention further contemplates the use of other currently known clinically relevant agents administered to treat the pathological conditions embodied in the present invention in combination with a compound of at least a molecule which binds IL-6 and/or the IL-6 receptor.

Moreover, the present invention contemplates that the compounds of at least a molecule which binds IL-6 and/or the IL-6 receptor are employed in either a treatment or prophylactic modality.

A preferred embodiment of the present invention is where the human to be administered a compound of the invention is female, and more preferred is when that human female is estrogen deficient.

Another preferred embodiment of the present invention is where the condition caused by an abnormally high level of C-reactive protein is cardiovascular disease, especially arteriosclerosis and thrombosis or other acute treatments in case of acute endothelial injury and/or destruction, like stroke, cardiac infarction, sudden cardiac death, burnt offering, severe surgery or other injuries with severe wound areas, diabetic shock, acute liver failure, pancreatitis, leucaemic persons after irradiation or long term endothelial injury and/or destruction, like arteriosclerosis, diabetes type I or type II, excessive body weight and/or obesity, alcoholism, Hormone Replacement Therapy (HRT), old persons, smokers.

A particularly preferred embodiment of the present invention is the use of a compound of at least a molecule which binds IL-6 and/or the IL-6 receptor in an estrogen deficient women, who is receiving estrogen or HRT, for the reduction of systemic or local inflammation.

Pharmaceutical formulations can be prepared by procedures known in the art, such as, for example, a compound of at least a molecule which binds IL-6 and/or the IL-6 receptor can be formulated with common excipients, diluents, or carriers, and formed into tablets, capsules, infusions and the like.

Examples of excipients, diluents, and carriers that are suitable for formulation include the following: fillers and extenders such as starch, sugars, mannitol, and silicic derivatives; binding agents such as carboxymethyl cellulose and other cellulose derivatives, alginates, gelatin, and polyvinyl pyrrolidone; moisturizing agents such as glycerol; disintegrating agents such as agar, calcium carbonate, and sodium bicarbonate; agents for retarding dissolution such as paraffin; resorption accelerators such as quaternary ammonium compounds; surface active agents such as cetyl alcohol, glycerol monostearate; adsorptive carriers such as kaolin and bentonire; and lubricants such as talc, calcium and magnesium stearate and solid polyethyl glycols. Final pharmaceutical forms may be: pills, tablets, powders, lozenges, syrups, aerosols, saches, cachets, elixirs, suspensions, emulsions, ointments, suppositories, sterile injectable solutions, or sterile packaged powders, depending on the type of excipient used.

Additionally, the compounds of at least a molecule which binds IL-6 and/or the IL-6 receptor are well suited to formulation as sustained release dosage forms. The formulations can also be so constituted that they release the active ingredient only or preferably in a particular part of the intestinal tract, possibly over a period of time. Such formulations would involve coatings, envelopes, or protective matrices, which may be made from polymeric substances or waxes.

The particular dosage of a compound containing molecules which bind IL-6 and/or the IL-6 receptor required to decrease levels of homocysteine and/or IL-6 according to this invention will depend upon the particular circumstances of the conditions to be treated. Considerations such as dosage, route of administration, and frequency of dosing are best decided by the attending physician. Generally, an effective minimum dose for oral or parenteral administration of a compound of molecules which bind C-reactive protein is about 1 to 20000 mg. Typically, an effective maximum dose is about 20000, 6000, or 3000 mg. Such dosages will be administered to a patient in need of treatment as often as needed to effectively decrease levels of IL-6 and/or the unoccupied IL-6 receptor concentration and/or inhibit conditions or detrimental effects caused by an excess of IL-6.

The invention is further described by the following examples.

IL-6 and Increased Cell Death

Interleukin-6 (IL-6) induces molecules like C-reactive protein (CRP) and Type II secretory phospholipase A2 IIA (sPLA2 HA). sPLA2 IIA hydrolyses the sn-2-ester bond of phospholipids to produce free fatty acids and lysophospholipids (e.g. lysoPC). CRP binds lysoPC and subsequently complement (for example as the first complement protein C1q) binds CRP.

IL-6 induces sPLA2 IIA and CRP in cultered hepatic cells. The expression can be inhibited by addition of antibodies (AB) specific for IL-6. A typical experiment will give the following results.

TABLE 1
Expression of sPLA2 IIA and CRP from hepatic cells after
induction by IL-6. Addition of antibodies specific for
IL-6 will inhibit the expression of CRP and SPLA2 IIA.

| | Expression of | Expression of |
|---|---|---|
| Conditions | sPLa2 | CRP |
| Hepatic cells | No | No |
| Hepatic cells + IL-6 | Yes | Yes |
| Hepatic cells + IL-6 | Yes | Yes |
| with control AB | | |
| Hepatic cells + IL-6 | No | No |
| with AB against IL-6 | | |

IL-6 and Atherosclerosis

Angiotensin II type 1 (AT1) receptor activation is involved in the development and progression of atherosclerosis. Stimulation of cultured rat aortic vascular smooth muscle cells (VSMCs) with IL-6 leads to upregulation of AT1 receptor mRNA and protein expression, as can be assessed by Northern and Western blot experiments. Blockade of IL-6 by antibodies specific for IL-6 or the IL-6 receptor decrease expression of the AT1 receptor.

Treatment of C57BL/6J mice with IL-6 for 18 days increases vascular AT1 receptor expression and enhances vascular superoxide production. These effects are strongly reduced by treatment with specific antibodies against IL-6.

TABLE 2
Expression of AT1 and enhanced superoxide production
in C57BL/6J mice after treatment by IL-6. Addition of
antibodies specific for IL-6 or the IL-6 receptor will
inhibit the expression of AT-1 and superoxide.

| | Expression of | Expression of |
|---|---|---|
| Treatment | AT-1 | superoxide |
| Control mice | Normal | Normal |
| Mice + IL-6 | Enhanced | Enhanced |
| Mice + IL-6 with | Enhanced | Enhanced |
| control AB | | |
| Mice + IL-6 with AB | Reduced | Reduced |
| against IL-6 | | |
| Mice + IL-6 with AB | Reduced | Reduced |
| against IL-6 receptor | | |

IL-6 and Reperfusion

In vivo experiments can directly show the relevance of sPLA2 HA in reperfusion injury. In rats, myocardial infarction and reperfusion can be mimicked by a brief artery occlusion. The size of the infarcted area can be determined. Addition of IL-6 will enlarge this area, while addition of antibodies specific for IL-6 will reduce this effect. Deposition of CRP will also be enhanced by IL-6, respectively reduced by specific antibodies. A typical experiment will give the following results.

TABLE 3
Effect of IL-6 and specific antibodies on infarct size and
deposition of CRP in reperfused rat hearts. The size of
the infarcted area in rats without IL-6 was set to 1.

| Conditions | Infarct size | Deposition of CRP |
|---|---|---|
| Control animals | No | No |
| Ischemia and reperfusion | 1 | Low |
| Ischemia and reperfusion with | >1 | Strong |
| IL-6 | | |
| Ischemia and reperfusion with | >1 | Strong |
| IL-6 and control AB | | |
| Ischemia and reperfusion with | 1 | Low |
| IL-6 and AB against IL-6 | | |

IL-6 and Inflammation

In another in vivo experiment, inflammation can be induced in mice by the injection of zymosan into the peritoneum. Inflammation will result in increasing serum levels of IL-6, sPLA2 HA, and SAP (the mouse equivalent for human CRP). The amount can be quantified in blood samples using ELISA techniques. Mice treated with antibodies to IL-6 will have lower sPLA2 IIA and lower SAP serum level than mice treated without these antibodies or with unspecific antibodies.

IL-6 and Wounds

Interleukin-6 (IL-6) is secreted in response to major abdominal operations. This leads to the recruitment of monocytes to the wounds. In mice the amount of monocytes attracted to the wound can be determined. Antibodies to IL-6 or the IL-6 receptor will decrease the number of attracted monocytes, lead to less inflammation and accelerated wound healing. Unspecific antibodies will have no influence on these parameters.

IL-6 and Interaction with the Immune System

Interleukin-6 (IL-6) leads to proliferation and maturation of B cells, as can be shown by IgM secretion. Activated endothelial cells (EC) produce IL-6. B cells cultered in supernatants from activated endothelial cells will start proliferation and maturation. Both can be blocked by antibodies specific for IL-6. A typical experiment will give the following results.

TABLE 4
Effect of supernatants from activated endothelial cells and antibodies
specific for IL-6 on proliferation and maturation of B cells.

| | IL-6 | | |
|---|---|---|---|
| Conditions | content | Proliferation | Production of IGM |
| B cells | No | No | Low |
| B cells with SN | No | No | Low |
| B cells with SNA | Yes | Yes | Yes |
| B cells with SN and | Blocked | No | Low |
| AB against IL-6 | | | |
| B cells with SNA and | Blocked | No | Low |
| AB against IL-6 | | | |
| B cells with SNA and | Yes | Yes | Yes |
| unspecific AB | | | |

(SN = supernatant from EC; SNA = supernatant from activated EC)

1-15. (canceled)

16. A method of treating or preventing a disease involving endothelial inflammation and selected from the group consisting of stroke, cardiac infarction, atherosclerosis with unstable angina, acute liver failure, hormone replacement therapy (HRT), and an immune disorder, wherein the immune disorder is selected from the group consisting of radiation-induced leukemia, allograft transplant rejection, xeno-transplant rejection, autoimmune disease, autoimmune liver disease, osteoarthritis, Graves' disease, dilated cardiomyopathy, Morbus Bechterew, inflammatory bile disease, ulcerative colitis, autoimmune thyroiditis, and myasthenia gravis, the method comprising reducing IL-6 concentration or unoccupied IL-6 receptor concentration by administering to a patient in need thereof a medicament comprising a therapeutically effective amount of an antibody containing an antigen-binding site for IL-6 or the IL-6 receptor.

17. The method of claim 16, wherein the IL-6 and IL-6 receptor are human.

18. The method of claim 16, wherein the antibody is a monoclonal antibody.

19. The method of claim 16, wherein the antibody is a recombinantly produced monoclonal antibody.

20. The method of claim 16, wherein the antibody is a recombinantly produced monoclonal, single chain scAb or scFv antibody, a bispecific antibody, or a diabody.

21. The method of claim 20, wherein the antibody is cross-reactive.

22. The method of claim 16, wherein the medicament further comprises a pharmacologically acceptable carrier or diluent.

23. The method of claim 16, wherein the medicament further comprises an anti-inflammatory substance selected from the group consisting of C-reactive Protein 9CRP) antagonists, CRP binding molecules, anti-IL-1β-molecules, PLA1 antagonists, PLA2 binding molecules, complement blockers, and combinations thereof.

24. The method of claim 16 for treating an immune disorder excluding rheumatoid arthritis.
